(19) United States
(12) Patent Application Publication
(10) Pub. No.: US 2023/0410956 A1
(43) Pub. Date: Dec. 21, 2023

(54) DATA STRUCTURE SUPPORTING CLINICAL STUDY CREATION, DISTRIBUTION AND FISCAL COMPONENTS

(71) Applicant: Exact Botanicals LLC, Coral Springs, FL (US)

(72) Inventors: Jaron B. Gladstone, Coral Springs, FL (US); Albert J. Angel, Coral Springs, FL (US)

(21) Appl. No.: 18/209,250

(22) Filed: Jun. 13, 2023

Related U.S. Application Data
(60) Provisional application No. 63/351,465, filed on Jun. 13, 2022.

(51) Int. Cl. G16H 10/20; G16H 40/20; G16H 10/60; G16H 15/00; G06Q 30/0207

(52) U.S. Cl. CPC G16H 10/20; G16H 40/20; G06Q 30/0215; G16H 15/00; G16H 10/60

(57) ABSTRACT

A data structure (DS) supports clinical studies and reports (CLS) by enhancing and coordinating creation of CLS, distribution of CLS (both internal by registered users (clinicians, patients, fiscal participants) and external/public. DS facilitates fiscal support for CLS and fiscally rewards registered-users with effort-based participation points for the clinical studies. Smart contracts on blockchain or DLT rewards registered-users for CLS comments, edits, internal and external distribution efforts logged into the blockchain. If successful (per block-contract), revenue from CLS distributed based upon point conversion.

This non-provisional patent application is based upon and claims the benefit of provisional patent application Ser. No. 63/351,465, filed Jun. 13, 2022, now pending, the contents of which is incorporated herein by reference thereto.

### FIELD OF THE INVENTION

The present invention is a data structure which supports one or more clinical studies enhancing and coordinating the creation of such clinical studies, the distribution of the clinical studies (both internal to registered users, clinicians, participants, fiscal participants, and external to the public at large), and provides a data structure facilitating fiscal support for the clinical studies. In one embodiment, smart contracts on a blockchain or distributed ledger technologic (DLT) system rewards clinicians, clinics, and patients who participate in clinical studies, and then further rewards clinical reviewers, follow-on patent participators, further clinical studies, and funders who support the clinical reports. If the clinical study is a success (success measured and monitored by the smart contract on the NFT blockchain), based upon public response to the publicly published clinical study report, the NFT blockchain is closed with, if available, a revenue distribution from sale of the study or the underlying study data. The registered users, clinicians, participants and fiscal participants are rewarded by distribution of funds related to the published clinical study.

### BACKGROUND OF THE INVENTION

It is well known that clinical studies are regularly conducted to determine the efficacy of drugs, drug regimens, medical devices, and other non-medical items, such as botanical compounds, nutritional compounds, and components. Society benefits from all these clinical studies. Although reference is made herein the clinical studies conducted by healthcare professionals, the invention is not limited to human studies nor healthcare professionals. The reference to “patients” applies equally to animals because such animals are subjects of the clinical studies.

One longstanding issue is how to increase patient participation in the clinical study. Many patients are not compensated for participating in such studies and, as a result, some of the patients may not regularly engage with the clinicians operating and conducting the clinical study. This is particularly true regarding botanical and nutritional compounds and compositions.

Another issue is funding studies at intermediate times during the patient data collection and prior to the final release of the clinical study report.

### SUMMARY OF THE INVENTION

The present invention monitors patient participation and, if the clinical study deemed to be success, based upon objective and demonstrable guidelines, patients who participate and engage in the study are fiscally rewarded. Of course, it is often times difficult to obtain fiscal support for clinical studies. The present invention provides a structure and a framework to enable fiscal participants to support studies and, if the clinical study is deemed a success, fiscally rewarded. Although success is sometimes difficult to determine, by providing additional incentives, such as participation points, to participants engaged in the study when those participants review a private version of the clinical study, provide edits and comments to that private or internal clinical study, the quality and success of the study and the underlying abstract for the clinical study report improves. In addition, by providing further participation points to engaged participants who aggressively publicly distribute and comment on public version of the clinical study, this participation reward further increases probability of success. Success may be measured in several manners such as by sale of the clinical study report, collecting fees for accessing the online public clinical report, and selling the underlying raw data (which has been scrubbed of personal identifying information PII) to other companies in the field of study.

It is an object of the present invention to provide incentives for patient participation in clinical studies.

It is another object of the present invention to provide, in some cases, fiscal reward not only to fiscal participants but also patient participants, clinicians who gather the patient data for the report, drafted the report, edit the report, and publish the report.

It is a further object of the present invention to provide, in some cases, fiscal reward to participants who promote both internally and externally (an external event being classified as a public event) by distributing the initial or private clinical study report and also distributing the follow-on public study report on a public data structure, such as the Internet.

Although the invention is illustrated and described herein as embodied in a data structure supporting clinical study creation, distribution and fiscal components, it is, nevertheless, not intended to be limited to the details shown because various modifications and structural changes may be made therein without departing from the spirit of the invention and within the scope and range of equivalents of the claims. Additionally, well-known elements of exemplary embodiments of the invention will not be described in detail or will be omitted so as not to obscure the relevant details of the invention.

Other features that are considered as characteristic for the invention are set forth in the appended claims. As required, detailed embodiments of the present invention are disclosed herein; however, it is to be understood that the disclosed embodiments are merely exemplary of the invention, which can be embodied in various forms. Therefore, specific structural and functional details disclosed herein are not to be interpreted as limiting, but merely as a basis for the claims and as a representative basis for teaching one of ordinary skill in the art to variously employ the present invention in virtually any appropriately detailed structure. Further, the terms and phrases used herein are not intended to be limiting; but rather, to provide an understandable description of the invention. While the specification concludes with claims defining the features of the invention that are regarded as novel, it is believed that the invention will be better understood from a consideration of the following description in conjunction with the drawing figures, in which like reference numerals are carried forward. The figures of the drawings are not drawn to scale.

Before the present invention is disclosed and described, it is to be understood that the terminology used herein is for the purpose of describing particular embodiments only and is not intended to be limiting. The terms “a” or “an,” as used herein, are defined as one or more than one. The term “plurality,” as used herein, is defined as two or more than two. The term “another,” as used herein, is defined as at least a second or more. The terms “including” and/or “having,” as used herein, are defined as comprising (i.e., open language). The term “coupled,” as used herein, is defined as connected, although not necessarily directly, and not necessarily mechanically. The term “providing” is defined herein in its broadest sense, e.g., bringing/coming into physical existence, making available, and/or supplying to someone or something, in whole or in multiple parts at once or over a period of time.

In the description of the embodiments of the present invention, terms such as “first”, “second”, “third” and so on are only used for descriptive purposes, and cannot be construed as indicating or implying relative importance. It should be noted that, unless otherwise clearly defined and limited, terms such as “coupled”, or “connected” should be broadly interpreted, for example, it may be electrically or functionally connected (such as with software); it may be directly connected, or may be indirectly connected via an intermediate medium or one or more software or program modules. The terms “program,” “software application,” “APP,” “software” and the like as used herein, are defined as a sequence of instructions designed for execution on a computer system. A “program,” “computer program,” or “software application” or APP may include a subroutine, a function, a procedure, an object method, an object implementation, an executable application, an applet, a servlet, a source code, an object code, a shared library/dynamic load library and/or other sequence of instructions designed for execution on a computer system. Those skilled in the art can understand the specific meanings of the above-mentioned terms in the embodiments of the present invention according to the specific circumstances.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require at least one of X, at least one of Y, and at least one of Z to each be present.

### BRIEF DESCRIPTION OF THE DRAWINGS

The accompanying figures, where like reference numerals refer to identical or functionally similar elements throughout the separate views and which together with the detailed description below are incorporated in and form part of the specification, serve to further illustrate various embodiments and explain various principles and advantages all in accordance with the present invention. Further objects and advantages of the present invention are found in the detailed description and in the accompanying drawings.

FIG. 1 diagrammatically illustrates the prior art clinical study (CL) process. The Abbreviations Table, near the end of the detailed description of the invention, provides general information about the abbreviations in the drawings.

FIG. 2A diagrammatically illustrates an outline of the blockchain clinical study.

FIG. 2B, which is a continuation of FIG. 2A, diagrammatically illustrates further aspects of the blockchain clinical study.

FIG. 3A diagrammatically illustrates a functional diagram of the participation point (PT) compiler.

FIG. 3B, which is a continuation of FIG. 3A, diagrammatically illustrates the point compiler function as a points conversion table, that is, the point-to-unit owner interest conversion table.

FIG. 4 diagrammatically illustrates the blockchain NFT for content and participation.

FIG. 5 diagrammatically illustrates the blockchain for curated content (CNT).

### DETAILED DESCRIPTION

The present invention is a data structure which supports one or more clinical studies enhancing and coordinating the creation of such clinical studies, the distribution of the clinical studies (both internal to registered users, clinicians, participants, fiscal partners, and external to the public at large), and provides a data structure facilitating fiscal support for the clinical studies.

The present invention relates to a blockchain system with smart contracts to reward clinicians, clinics, and patients who participate in clinical studies, and then further rewards clinical reviewers, follow-on patent participators, further clinical studies, and funders who support the clinical reports. If the clinical study is a success, success being monitored and measured by the NFT blockchain system, then the clinical study report is publicly published and the NFT blockchain is closed with, if available, distribution of revenue from the study. Some abbreviations used in the drawings are set forth in the Abbreviations Table near the end of this specification.

The invention employs a Web 3.0 format and data structure to provide a collaboration among persons and institutions (often unaffiliated with each other) for medical and clinical research. Today's clinical study environment is siloed with pharma companies keeping clinical study data and knowledge close to the vest. A blockchain collaboration allows decentralized, collaborative associations, with contributions by diverse participants working toward a project conclusion that is beneficial to scientific progress and beneficial to the contributing collaborators. For example, multiple clinical studies can be carried out by clinics which are widely geographically separated. The smart contract, blockchain format of the present invention keeps both clinical study (CL) data and data and engaged party activities (those who participate in the clinical study CL) well organized and functional in a Web 3.0 way. To the extent these collaborations get formed, they can absorb financial contributions, research and operational contributions, including an administration among diverse, but like-minded participants.

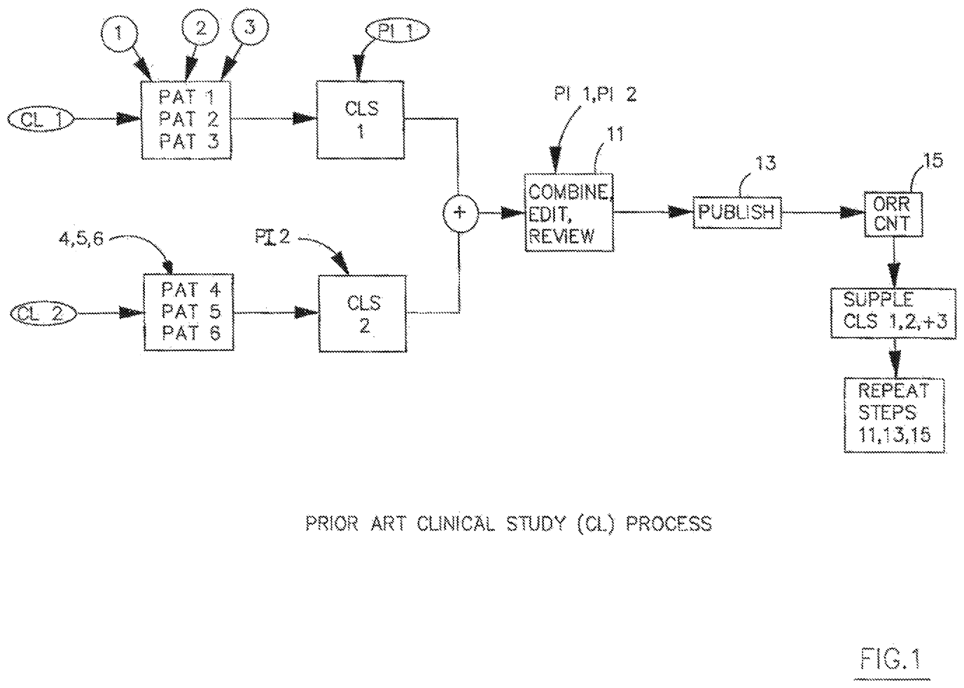

FIG. 1 diagrammatically shows the process flow for a clinical study (CL) process as a prior art process. FIG. 1 shows a pre-existing process wherein patients 1, 2, 3 (PAT) participate in clinical studies (CLS) and, more particularly, engage with two different clinics CL1 and CL2. Clinicians participate by operating the CL facility and collecting data from the participating patients. Principal investigators (PI), namely PI 1 and PI 2, are the leaders of the clinical study, CLS 1 and CLS 2. Data from CLS 1, 2 is combined at the summation point. PI 1 and PI 2, in addition to other subordinate clinician participants, review the data from CLS 1, 2, analyze the data, and draft a clinical study report (CLS). Preparing the clinical study report CLS is diagrammatically illustrated in step 11. Principal investigators PI-1, PI-2, develop the CLS report and, in step 13, publish the CLS report. Publication, in this prior art system, involves distributing the CLS report publicly. Outside review and critical commentators ORR provide content (CNT), which may be complementary or critical of the CLS report, and generally provide supplemental material to the clinical study reports CLS 1, 2, and 3. Prior art clinical studies continually repeat steps 11, 13 and 15.

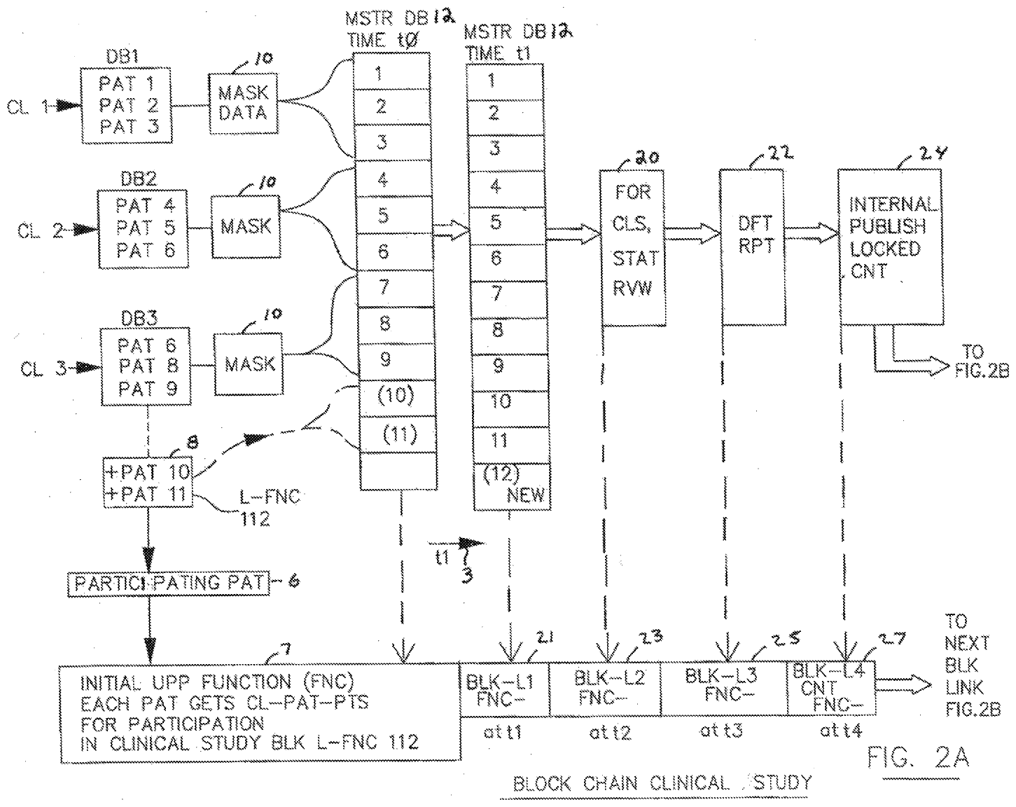

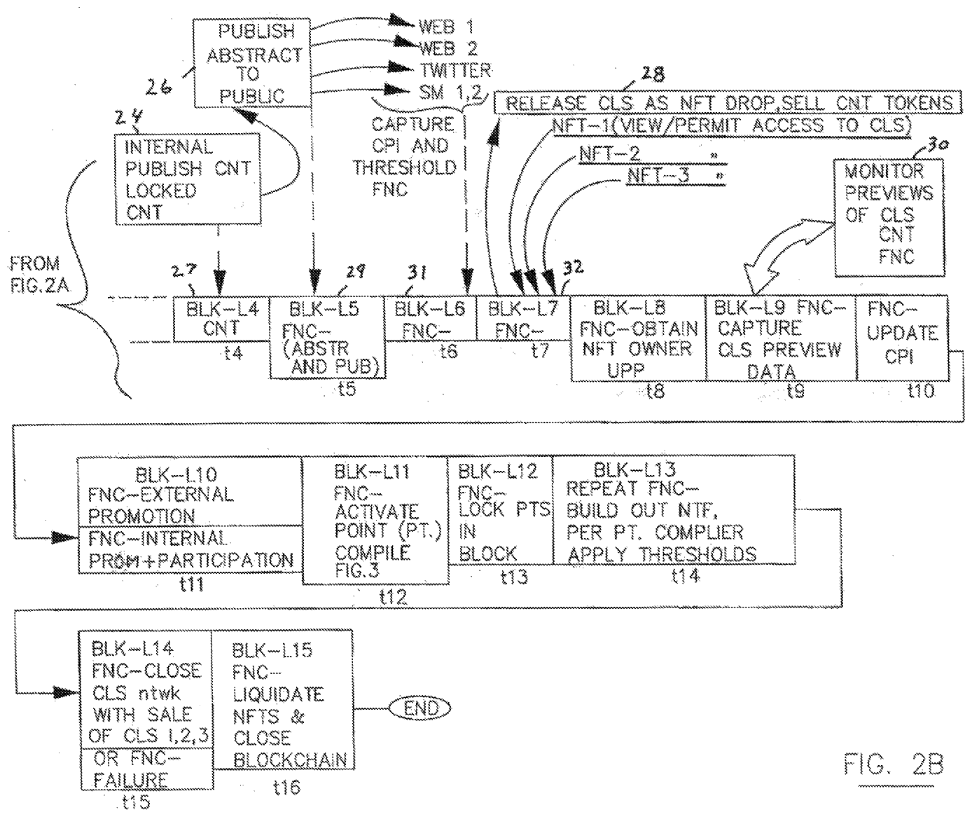

FIGS. 2A and 2B diagrammatically show aspects of one embodiment of the present invention Clinical Study Report (CLS Rpt) Process wherein participation and promotion of the patient participants (PAT) and the clinicians involved in the clinical studies generating clinical study reports (CLS) are rewarded via a blockchain NFT process. FIGS. 2A and 2B are related by a flow process. On the left side of FIG. 2A, clinicians at various clinical sites CL1, CL2, CL3, engage and interact with different groups of patients PAT 1, PAT 2, etc. as is common in prior art clinical studies. Data is gathered on these patients and stored first in databases DB1, 2, 3 and then, in a PII masked manner in the master database, Mstr DB 12 at time T-0. The mask function (FNC) is carried out in function block 10. Certain personal identifiable information PII is excluded from the PAT data in Mstr DB 12 for the to-be-created CLS report as noted by the mask data function 10. Functions in the NFT blockchain are abbreviated “FNC-”. These functions may be automated by smart contracts or programs in the blockchain. The clinicians at clinics CL 1, 2 and 3, upload PAT data to a master database 12 at the time T-0. As noted at a subsequent time T-1, this master database 12 is expanded to add new patients and new patient data PAT 10, and PAT 11. See event block 8. Event block 6 adds PAT 10, 11 data to the blockchain as participating patent data. Patient data PAT 10 and 11 is added to MSTR DB at time T-0 whereas PAT 12 data is added to DB12 at time T-1. Arrow 3 shows the timing sequence from T-0 to T-1.

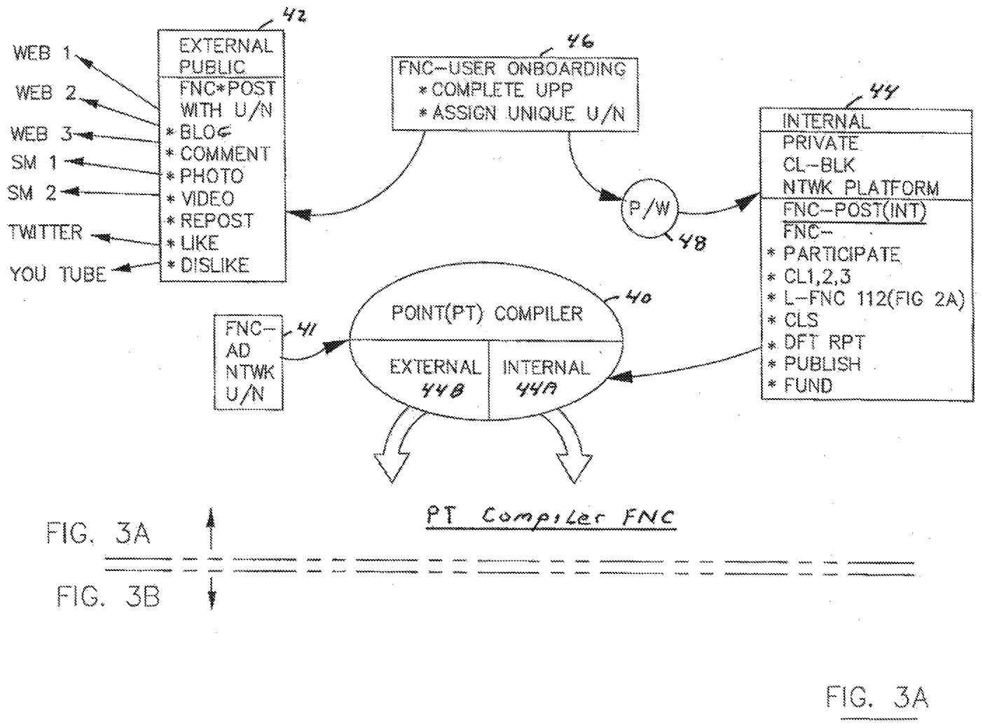

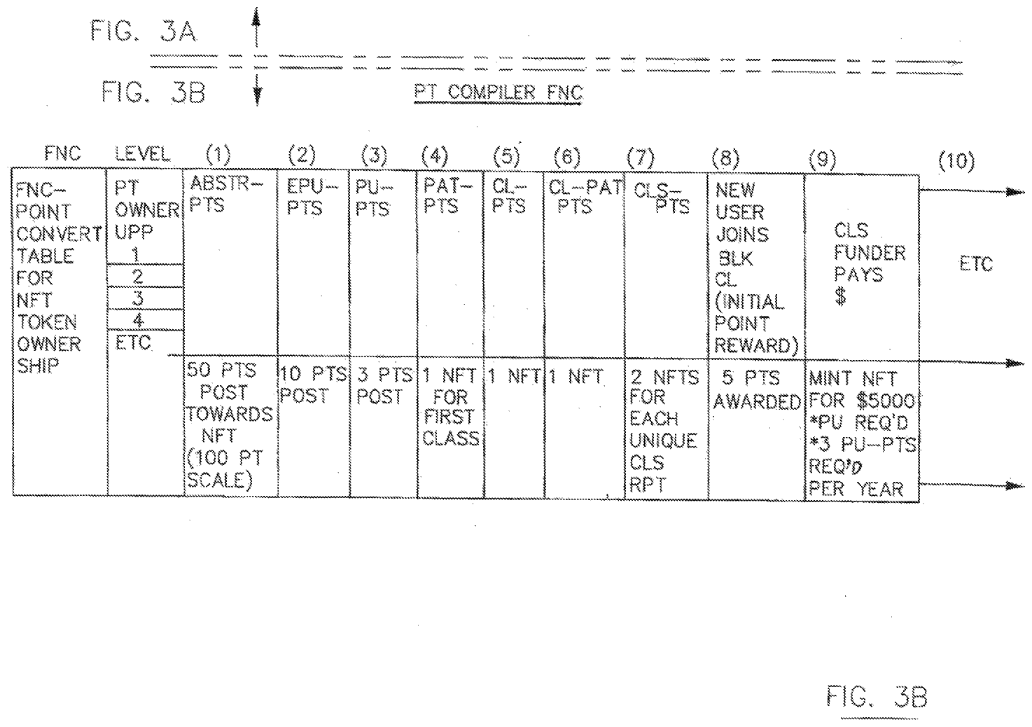

The blockchain function “L-FNC 112” at cell block 7 is discussed later in connection with FIG. 3A but refers to assignment of an initial patient participating points (Pts) to newly added PAT 12. There may be a referral point system (in addition to the point system discussed later) for referring PAT 12 to CL 3 clinical study wherein referral points are awarded to, for example, PAT 10 and PAT 11.

As for the blockchain in FIG. 2A (see cell blocks 7, 21, 23, 25 and 27), the initial blockchain or nonfungible token NFT is established by a blockchain developer. Cell 7 accepts initial user personal profile UPP data as function FNC, which, is loaded into the appropriate clinical study database DB1, DB2, DB3 and MSTR DB. A pass word, pass code or access control is assigned to the patient participator, non-patient participator, clinician or fiscal participant when he or she joins the blockchain clinical study. In this manner, as a generalization, when the master database 12 at the time T-1 is complete, a block 21 is entered into the blockchain as shown by the dashed lines leading from MSTR DB 12 at time T-1 to block 21. BLK-LI block at time T-1 enters a notation of completion of the data gathering step by the clinicians conducting the CLS study. Each PAT may have a “participation” stake or point(s) in the blockchain-NFT. Also each patient participator, non-patient participator, clinician and fiscal participant is assigned a participation point pt at cell 7.

Operationally, the blockchain includes one or more smart contracts which cooperate with oracles (data monitoring and input modules that gather data) and these embedded smart contracts in the blockchain include functions that generate the results described herein. The operation of the smart contract or contracts sets forth the agreement(s) or ground rules between collaborators interacting with the blockchain. It should be noted that the invention encompasses a high degree of flexibility in format, inputs, functional operations by one or more embedded smart contracts, and outputs. The descriptions of the operation herein are only exemplary of various operational aspects. For example, the financial outputs in FIG. 3 may be optional or not used in the basic clinical systems in FIGS. 2A and 2B. One smart contract might track and log contributed time in a measured quantitative way (e.g., 1 point per hour, a time-based participation tracker), whereas another smart contract might want to identify qualitative contributions, where, for example, a profound or pivotal contribution is made in the project, for which additional points are awarded to that contributor, or a weighted allocation is made. A DAO may be employed to determine allocation of reward points. The participants might provide in that smart contract that there will be an extra allotment of points for profound contributions, or original break-through insights, etc. and that those will be made by during and after-the-fact vote by the participants themselves, or alternatively by an independent entity that applies the smart contract rules, and assesses the contributions to make a determination.

Returning to FIG. 2A, the clinicians at facilities CL1, 2, 3 or a different group of clinicians (those persons creating the CLS content report as content (CNT) creators) begin reviewing the clinical study data CLS statistically in the “Stat review” event block 20. Another block 23 is entered in the blockchain at time T-2 to indicate the statistical review participation of the clinicians. An interim, draft version of the clinical study report or CLS report is prepared at event block 22 and the blockchain notes this completion at chain block 25, T-3. The block 25 in the NFT blockchain at the time T-3 documents the creative development of the draft and the input of the PIs and healthcare (HC) providers or clinicians into the CLS draft report (event 22). Again, the CLS Report drafters are given credit “convertible NFT ownership units” for drafting the CLS report. These convertible NFT points can be converted into NFT ownership tokens when the point owner reaches certain defined or predefined point thresholds. See FIG. 3A and 3B.

In FIG. 2A, event block 24, the CLS report is internally “published” as locked content (CNT) in the blockchain at BLK-L4 at time T-4 (NFT block 27). The term “locked” refers to an internal publication on the CLS NFT blockchain. It is known that the NFT system and blockchain programming can automate the creation of several blocks based upon data input and output in and to the master database 12 at T-0 and T-1 and can document events 20, 22 and 24 at T-2, T-3 and T-4 to document to development of the CLS report by the clinicians and for documenting and rewarding creation of various revisions of the CLS report. Once the CLS report is complete, it is subject to limited, internal publication on the NFT blockchain platform at BLK-L4 at time T-4. The term “BLK-L4 CNT FNC-” represents the NFT blockchain (BLK), link or cell L4, operating as an automated or smart contract to monitor and confirm the event. The “internal publication of CLS Rpt” in event 24 is documented in the NFT chain via the function (FNC-) in cell 27 which monitors internal publication of CLS report content CNT at time T-4. Although the term “published” is referenced in FIG. 2A, the publication is not made generally known to the public but is logged-in and posted for limited publication in the blockchain as a documented event in the blockchain at time T-4. It is known that blockchains and NFT's have password control functional elements. In this manner, clinicians who participate in the data gathering and others who participate in the drafting of the CLS report and financial or fiscal funders who assist clinicians in developing CLS report can access the locked CLS report in the blockchain at time T-4. Also, the NFT link or cell may have a pointer to access the CLS Report on another access controlled network when the report is stored on a different digital platform, different from the NFT platform.

FIGS. 2B repeats last process event (internal publication of the CLS report) and NFT link or cell BLK-L4 CNT FNC- which is shown in FIG. 2A.

Although the invention is particularly described above, the concept is not limited to NFT blockchains. The present invention is a method of operating a data structure to support a clinical study CL. A plurality of patients PAT participate in the clinical study and one or more healthcare clinicians conduct the study by engaging with and monitoring the clinical trial patients. Data is compiled in DB1, 2, and 3. Also, one or more fiscal participants (not specifically shown in FIGS. 2A, 2B) support the clinical study by individually fiscally supporting the client study. Each patient, of the plurality of patients, each clinician of the one or more clinicians, and each fiscal participant of the one or more fiscal participants, is sometimes referred to as an engaged participant. The participant engages in the CLS study at different times and has different roles.

The invention operates on an internal or controlled data structure and an external, data structure, sometimes referred to as a public data structure. One example of a public data structure is the internet. The internal or controlled data structure (block 44, FIG. 3A) has access controls (see password event 48, FIG. 3A) which limits access by only engaged participants (excluding non-engaged members of the general public). Access is provided for each engaged participant on the internal or controlled data structure. Prior art blockchains typically reside on password controlled data structures. Also, the engaged participants have access to an external public data structure (block 42, FIG. 3A), which typically is the Internet. However, the public data structure may be a private or limited access internet structure, much like a company-wide internet or cloud- based, limited access data structure. The inventive system and method assigns participation point (Pts) based upon individual efforts of each engaged participant. The system and method also assigns clinician Pts to each clinician of the one or more clinicians for compiling clinical study data (DB 1, 2, 3, FIG. 2A), and one or more of (i) drafting an abstract (the abstract is a preview of the CLS report and represents content CNT), (ii) drafting a clinical study report (events 20, 22, FIG. 2A), and (iii) editing the abstract and CLS report. The system and method further assigns fiscal Pts (FP) to each fiscal participant of the one or more fiscal participants based upon his or her individual fiscal support of the clinical study. See FNC Level 9, FIG. 3B.

In general, there are internal interactions (event block 42, FIG. 3A) and thereafter external interactions (event block 44, FIG. 3A) with the abstract and the clinical study report. Abstract points are shown in FIG. 3B, FNC Level 1. After an internal distribution of the abstract on the controlled data structure, the inventive system and method assigns an internal abstract participation point (APP) based upon individual efforts of engaged participants who have an abstract interaction (IAI). See internal events in block 44, FIG. 3A. The abstract interaction (IAI) can be one or more of an IAI comment, an IAI edit, an IAI criticism, or an IAI distribution act. Hence, engaged participants collect abstract APP points based upon some interaction on the controlled or internal data structure. See event block 44, FIG. 3A. After an internal distribution of the clinical study report on the controlled data structure, the system and method assigns an internal clinical study report point (RPP) to engaged participants based upon individual efforts of those engaged participants who have an internal clinical report interaction (IRI). The clinical report interaction (IRI) may be one or more of an IRI comment, an IRI edit, an IRI criticism, or an IRI distribution act. See internal events in block 44, FIG. 3A. The distribution interaction may be forwarding the abstract or the clinical study report to other engaged participants on the internal or access- controlled data structure. Emails or text messages with the internal report involve a distribution interaction.

Progressing from FIG. 2A to FIG. 2B, the internally published and locked-in CLS report (event 24), identified as content CNT, is then published as an abstract (Abstr) in event 26, FIG. 2B, on various medical websites Web 1, 2, and Web 3.0, as well as promoted on social media SM platforms such as Twitter and SM 1, 2 at time T-5. Event 24 and chain block 27 (BLK-L4 CNT) is duplicated in FIG. 2B from FIG. 2A in order to clarify the operational aspects of this specific embodiment. The abstract made be generated automatically by AI (for example, chatGPT or other LLM programs) by a function in the blockchain or may be drafted by the HC providers-clinicians who created the CLS report. The abstract is published on the external or public data structure in event 26 and is monitored and noted in chain block 29, BLK-L5 FNC, abstract publication. BLK-L6 Function chain block 31 monitors performance of the abstract on the public data structure. The number of individuals (i) accessing, (ii) reviewing, and (iii) responding in some online manner to the CLS abstract is monitored by the blockchain or NFT by capturing CPI data (clicks per view, typically 1,000 views is 1 CPI) from these abstract views (including followers, positive and negative review indicators (likes and dislikes)). The CPI data identifies the number of views, the number of unique viewers of CLS abstract, the number of followers, and number of individuals who share CLS abstract. Also, the blockchain cell 31 can capture re-tweets of the abstract, showing breadth and depth of the CLS abstract interest. This data is subject to a special CPI thresholding function FNC and the threshold modified data is captured and stored in the blockchain and BLK-L6 FNC- at time T-6. The blockchain also captures the initial publication of the abstract and public interactions at block BLK-L5 at intermediate time T-5.

Further in FIG. 2B, the CLS report is publicly released on the public data structure. In this specific embodiment, an NFT token is released, minted or dropped at the BLK-L7 FNC- block at the time T-7. NFT tokens 1, 2, 3 are minted at time T-7 to attract either other patients who may participate in follow-on clinical studies, or clinicians who may want to provide supplemental clinical study reports as well as CLS financiers. Further, in this specific embodiment, NFT ownership tokens can be minted for CLS patient participants, CLS content creators (that is, clinicians) and financial fiscal backers. “NFT-1” permits only viewable access to the CLS report. In this embodiment, only the abstract is publicly available as noted in event 26. NFT-1 is a view-only access link to the full CLS report, then stored on the access-controlled data structure. For example, patient participants and clinicians participants may have internal, access to CLS report which is only viewable on the access - controlled data structure. Fiscal participants also have access to the CLS report. Other members of the public do not have access CLS report. In some situations, public access to the CLS report may be a pay-per-view event. Each one of these actions or events generates another block, cell or link on the blockchain as a minted NFT token in this specific embodiment of the invention.

BLK-L8 Function obtains, at time T-8, for each member of the public who wishes to see the CLS report, PII data. This data is stored in the UPP database for the access-controlled data structure. Block BLK-L9 Function, at time T-9, monitors the number of views and interaction of the public with CLS report and the abstract interaction of the public in connection with the CLS abstract. This is event 30 which monitors previews of the CLS content. Function Update CPI, at time T-10, indicates that the blockchain is compiling data showing the importance (or lack of importance) of the abstract or the clinical study report (CLS report) in the public sphere.

Following the blockchain configuration of this specific embodiment to time T-11, Functions “FNC-” monitor the external promotion efforts by patients (PAT), clinicians, content CNT creators, ORR (outside reviewers, revisors or commentators), and financiers (fiscal participants), of the CLS report and the earlier published abstract. Also, the function at time T-11 documents internal participation by PAT and HC providers who drafted or participated in the CL studies. For example, if patients, who initially participated in the report, share the CLS report on social media and other websites, this external participation is documented in BLK-L10. As discussed later, the promoting PAT or participant or CNT creator is assigned points for each such promotional events. Internal participation in BLK-L10, time T-11, is, for example, further PAT follow-on studies, and published CLS report reviews by ORR (outside reviewers) on the internal, access-controlled data structure. Stated otherwise, if a participating patient discovers in outside or external review ORR of the CLS report and then copies or posts this external review on the access-controlled data structure, that participating patient or engaged participant is awarded additional points for this internal activity of posting and external review.

At time T-8, the NFT gathers UPP data on all new NFT token owners. At time T-9, the NFT captures data showing how many people view the now public CLS report. Typically, this is CPI data. At time T-10, the NFT updates the CPI data store and count. This CPI data store can be maintained on the blockchain or in the MSTR DB 12, FIG. 2A. At time T-11, BLK-L 10 monitors both external promotional events and internal promotional events. At time T-12, BLK-L 11 activates the point (pt.) complier discussed in connection with FIG. 3A.

At the time T-13, BLK-L12, points are locked or stored into the blockchain. At time T-13, BLK-L13, mints NFT tokens based upon the point compiler output and NFT point thresholds. The NFT thresholds are point conversion tables. Additional blocks are minted at BLK-L14. In other words, it may take some time for the preview of the CLS report to become known and become a matter of public interest. BLK-L13 contemplates that the smart contract in the blockchain cell L13 monitors the performance of the clinical study report and the monetization of the same. The pay-per-view initial setting may be altered dependent upon the performance of the clinical study reports based upon CPI or external comments or reviews or traction on Twitter or Facebook social media platforms. Ad revenue can also be monitored by the smart contract in the specific blockchain, cell L13 contemplates that monitoring, generation or minting of additional NFT tokens. Others who wish to review the CLS report may either (a) be a qualified ORR (a qualified ORR may be a person who wishes to become an engaged participant or a registered participant based upon his or her interest in the clinical study which is documented by the qualified person completing the user personal profile UPP by the access-controlled data structure) or (b) pay for a full version of the CLS report. At time T-15, the an NFT smart contract “close” function is activated which determines whether the clinical study report has garnered enough interest to result in the sale of the CLS report to some third party or, more particularly, the sale of CLS 1, 2 and 3 clinical data, and hence to qualify as a “success”. Alternatively, the clinical report may not have generated sufficient interest. Sufficient interest maybe: follow-up patient participation, or use/views on the Internet or social media, or NFT financing (see FIG. 3A), or other critical comments by other clinicians ORR (those ORR clinicians not drafting the original report). The result being that the NFT has failed to meet predetermined thresholds of success. If a failure event has been determined by the smart contract in NFT function (FNC-) at cell L14, time T-15, the smart contract initiates a “failure” function and closes down the NFT system without providing any financial benefit to the participants, content creators, promoters and financiers. In the event of a success determination from FNC L14, cell block BLK-L15, at time T-16, the function liquidates the NFT, based upon the success event, and any funds gathered from the CLS report are distributed at time T-16 to the NFT token owners. Sometimes NFT token owners are referred to as having an ownership interest unit OIU. The following Close Function Table provides some examples of thresholding events to trigger the close function.

Close Function (FNC) Table
Time-Based (e.g., 1 yr, or 18 mths, etc.)
Patient Count
CLS Rpt high statistical efficacy
CLS Rpt high market response
Acceptable Offer to sell CLS data and CLS Rpt
Patient Data UPP Access (possible
delete PII data from CL DB)
DAO Threshold event
NTF Owner Consensus
Failure Event: Threshold a, b, or c not met

In a more general sense, after the internal interactions, the system and method releases, to the public data structure, a public version of the abstract which is “subject to” the internal distribution of the abstract. The abstract may be a preview of the formal CLS report. For example, the internal distribution may suggest changes to the abstract based upon comments, edits, or criticisms. These internal interactions are monitored and participation points Pts are awarded to the interacting parties. Once the abstract or preview is on the public data structure (which may be the Internet or limited private network), engaged participants (sometimes called registered participants) are assigned external abstract participation points (xAPP) based upon individual efforts of those engaged participants who have external abstract interactions (EAI) with the public abstract. These EAIs can be comments, edits, criticisms, or an EAI distribution act. After the public abstract is released, a public clinical study report is released. Hence, the system and method releases to the public data structure the public clinical study report subject to the internal distribution of the clinical study report. Again, the public clinical study report may include edits or responses to internally created content (CNT) comments, edits, or criticisms. External report participation points (xRPP) are assigned the interacting parties based upon individual efforts of the engaged participants who have an external report interaction (ERI), that is an ERI comment, an ERI edit, an ERI criticism, or an EM distribution act.

The system and method assigns either prior to, during, or after the clinical study, or whenever a fiscal participant engages to fiscally support the clinical study, an ownership interest unit (OIUs), from a plurality of ownership interest units, to the engaged fiscal participant (who may be one of many fiscal participants) on a fiscal pro rata basis. The system may cap the number of OIUs, or not, dependent upon a scarcity characteristic determined prior to the launch of the CLS data structure. A DLT data structure and a DAO data structure can be configured in a similar manner. In other words, a unit of ownership is assigned to fiscal participants based upon their respective financial investment in the clinical study venture.

In addition to ownership interest units OIUs assigned to fiscal participants, the points garnered by engaged/registered participants, clinicians, and clinical patients are converted into one or more ownership units based upon a predetermined point-to-unit conversion. The points are participation Pts, clinician Pts, fiscal Pts, APP, RPP, xAPP, and xRPP. In this manner, the fiscal participants are rewarded if the clinical report is favorable, and the patients, the clinical participants, and all other engaged participants are rewarded and hence motivated to collect more points by participation, drafting, editing, reviewing, commenting on, and sharing the abstract as well as the internal and the external or public discussions and further distribution (internally and externally on the public data structure (possibly the Internet)) and are rewarded by assignments of one or more ownership units OIUs based upon a predetermined point-to-unit conversion.

A smart contract, operating on the controlled data structure and on the public/internet data structure, determines success of the public clinical study report by monitoring performance interest on the public data structure (CPIs and social media indicators) when performance interest exceeds a predetermined threshold. Performance interest can be measured in several ways, such as by cpi (clicks per 1,000 views), offers to buy, critical commentary, laudatory commentary, etc. If the preprogrammed smart contract determines success, then engaged/registered participants are fiscally rewarding based upon their ownership units. Points are converted to OIUs by a conversion plan and program.

Further enhancements include assigning patient participation points (PPP) to each patient for their individual effort in the clinical study, and, to reward clinicians, the clinician Pts are differentiated from engaged participant Pts and from PPP. Fiscal Pts (FP) are also differentiated from both clinician Pts and patient participation points PPP. In this manner, work by clinicians is rewarded at a higher level than patient Pts. The controlled data structure is communicatively coupled to the internal data structure and the public data structure to conduct monitoring and distribution. This may be an autonomous operation. All functions may be autonomous.

As such, the controlled data structure may include one of a blockchain, a non-fungible token (NFT) blockchain, and a distributed ledger technologic (DLT) structure. Prior to granting access to the controlled data structure, engaged participants provide personal identifying information (PII) to a user personal profile (UPP) data store on the controlled data structure and access to the controlled data structure is provided by a respective access pass assigned to each engaged participant. Also, non-participants may sign up with the controlled data structure. The plurality of non-participants who are interested in the clinical study are sometimes called incipient engaged participants. The system and method gathers PII from the incipient engaged participants for the UPP data store. Once the PII is uploaded to the UPP data store, each incipient engaged participant defined as an engaged or registered participant.

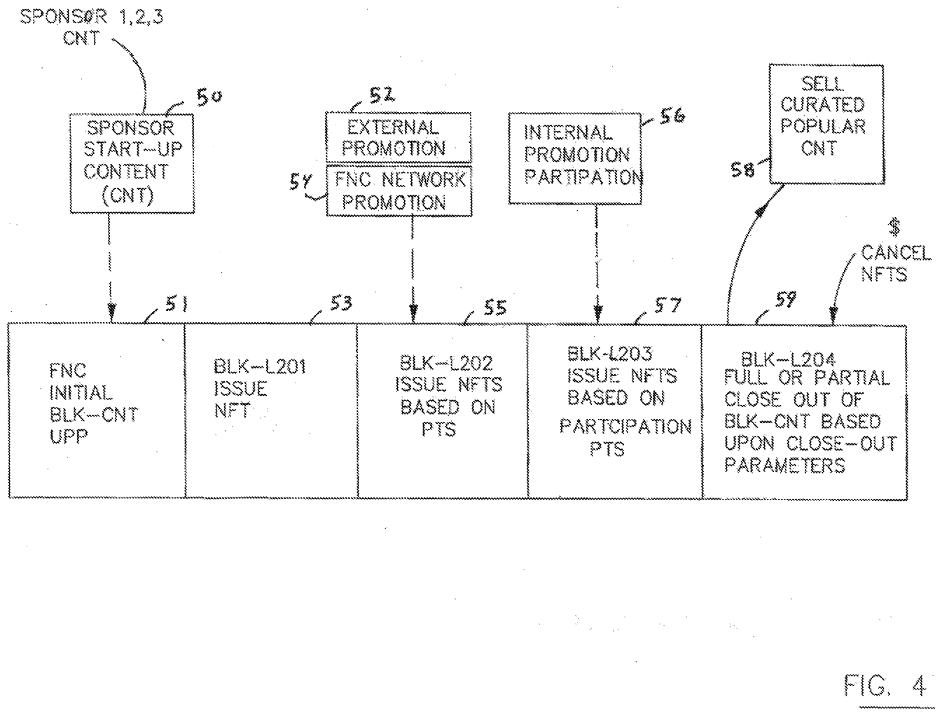

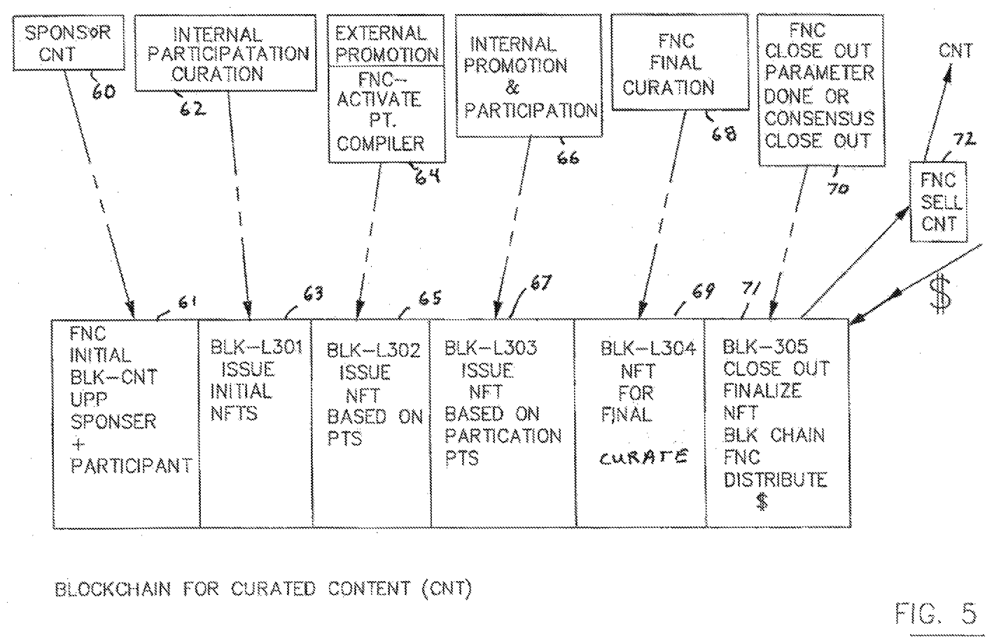

As another enhancement of the invention, interaction with an engaged participant is rewarded by assigning a general participation point (GPP) to each engaged participant: (m) who interacts with the internally distributed abstract, (n) who interacts with the internally distributed clinical study report, (o) who interacts with the public abstract, and (p) who interacts with the public clinical study report. The a general interaction (GI) is one or more of a GI comment, a GI edit, a GI criticism, or a GI distribution act. The system differentiates GPP from clinician Pts, patient participation points PPP, and fiscal Pts (FP) because the GI interactions are less valuable to the success of the CLS study. However, GI interaction rewards, which are open to the public, do advance the success of the overall CLS program. The conversion of participating Pts into one or more ownership units OIUs includes converting GPP points into one or more OIUs based upon the predetermined point-to-unit conversion. The concept employed by the present invention is that the point system, which ultimately translates into OIU ownership claims can be varied based upon: (a) the skill level of the person delivering the service to the CLS data structure; (b) the CLS demand for patients suffering from certain conditions or having a condition within the bounds of the CLS study (that is, a supply of patients who generate useful data); and (c) the time and effort expended by the participating person. Understandably, factors 1 and 3 are on a sliding scale and the point system presented herein cannot cover nor address all CLS studies, or, for that matter, all curated content (CNT) as discussed in connection whit FIGS. 4 and 5. Example (1): a skilled clinician working in the CL clinic has more time and effort than the patient at the clinic and is highly valuable and needs compensation and recognition (in a similar manner to fiscal participants, who are also essential). Example (2): given certain guidelines, any member of the public who expresses an interest in the CLS process may want to be a visiting participant and not an overtly active participant, and hence that visitor should be given some recognition and participation points notwithstanding he or she is doing much less than patients, clinicians, report drafters and editors. The CLS data structure, as a whole, benefits from these limited participating visitors because, when the CLS report becomes public, these visitors may greatly assist in the sale of the CLS report or the underlying data.

Another configuration of the system and the method is a data structure supporting a clinical study by monitoring patient participation (patient Pts or PPP), assigning points for the clinical study (that is, giving points to the clinicians (CPP points) gathering patient data, then drafting the abstract, then drafting the clinical study report, responding to suggestion and criticisms, etc. as explained above. Patient participation points (PPP) are assigned to each patient who participates. Clinician participation points (CPP) are assigned to each clinician who operates the clinical study, creates an abstract, creates the clinical study report. All this is based upon the respective clinician participation level. Fiscal points (FP) are assigned to each fiscal participant based upon individual's fiscal support of the clinical study. Other points are: internal abstract participation (APP points); internal abstract interaction (IAI points); internal report participation points (RPP); external abstract participation points (xAPP) (EAI interactions); external report participation points (xRPP) (based on ERI comments, edits, criticisms, and ERI acts of distribution). Ownership interest units OIUs are assigned fiscal participants based on FP points (essentially on a fiscal pro rata basis). The various Pts (PPP, CPP, FP, APP, RPP, xAPP, and xRPP) are converted into ownership units OIUs based upon a predetermined point-to-unit conversion plan. Further enhancements include: assigning an additive clinician participation point (xCPP) to each clinician (w) who interacts internally with abstract, (x) who interacts internally with clinical study report, (y) who interacts publicly with the abstract, and (z) who interacts publicly with clinical study report. The clinician interaction (CI) is mapped or based on CI comments, edits, criticisms, and CI distribution acts. The fiscal participation points FP may be assigned pre or post assignment of PPP points to patients or assignment of CPP to each clinician.

Further aspects of the invention include (a) uploading each patient's clinical study data into the controlled data structure (b) providing access from the controlled data structure to a public internet data structure (Internet) via a data communications module. The communications module is a communicative bridge function permitting certain blockchain initiated operations from the controlled data structure to be executed on the Internet or the public data structure. In other words, the smart contracts on the blockchain can monitor actions on the public data structure (typically, the Internet).

An important computer and data structure aspect of the present invention is to solve the problem of interim financing, that is, prior to the final public release of the CLS report. Fiscal participants cannot see or review interim clinical study data. However, the present data structure does permit internal, interim reports to be viewed by fiscal participants on a confidential basis. Patient data is masked by striping out PII. Interim CLS reports are internally circulated and subject to comment and criticism. Fiscal partners could review this data structure and decide to fund or not to fund when called upon by the clinical team. Therefore, the present invention is an improved computer data processing structure and method.

FIGS. 3A and 3B diagrammatically show a flow chart and system diagram for collecting and monitoring participation and promotion points rewarded via a blockchain NFT process. FIGS. 3A and 3B are linked together as noted by the reference to the Point (PT) Complier Function (FNC) in both figures. FIG. 3A is the system or process for the point calculator or point compiler 40, PT Compiler 40. Points may result from an external action event 42 or an internal action event 44 (internal typically referring to the specific NFT embodiment discussed earlier and the NFT-centric activities such as drafting CLS reports, participating in follow-up studies, posting comments on the NFT website or nonpublic actions on the NFT platform). As explained earlier, the inventive method and system can be built on many types of data structures, DLT systems and DAO systems, as well as internal, non-public networks. Point Compiler Function 40 monitors internal and external interactions.

FIG. 3A shows, in this specific NFT embodiment, that points can be earned by various interactions. Once a participant becomes an engaged (registered) participant by completing event function 46 and uploading his or her PII to buildout the participants' user personal profile UPP, the system assigns a unique user name U/N to that engaged participant and assigns a password or passcode or access tool as shown by the event 48. The access tool or code permits the engaged participant to interact as shown by internal event 44. Events in event set 44 are generally monitored by point complier 40 and specifically by internal compiler 44A. As discussed above, in this specific embodiment, a CL-BLK data platform is established which only permits access to the assigned access tool. Internal Event 44 recognizes an internal posting event (with a monitoring function), a general participation event, and patient participation in CL 1, 2, 3 studies. The assignment of an initial number of points for joining the clinical study network and shown in FIG. 2A, Block cell 7, L-FNC 112, wherein, upon joining the network, the engaged party is assigned an initial amount of points. As discussed, engaged participants interacting with the CLS reports, drafting the content (CNT), publishing internally the abstract, and providing fiscal funding to the clinical study all results in the assignments of points to that engaged participant.

FIG. 3A also shows, for external event 42, points for posting when the engaged participant uses his or her assigned user name U/N when he or she interacts with blogs, posting comments on various webpages and social media including twitter and YouTube, posting photographs, video, reposting, assigning likes and dislikes to the external public abstract and to the external public CLS report. This results in a further assignment of points. In connection with these external activities, the point compiler program 40 monitors performance on the internal and external data structures (the later sometimes being the Internet) by tracking the user name assigned to the engaged participant. External events are compiled by point compiler 40 as noted in external compiler monitor 42A. Internal points are compiled and monitored by internal compiler 44A. Event 41 also monitors actions on the external/Internet data structure by way of the ad network which tracks user names.

The system has a function FNC 42A monitoring the posting of online data by monitoring UPP-registered users (those having system user names) and logs user-generated comments, photos, video, reposting acts, likes, and dislikes. Most likely, given the clinical nature of the studies, a private web site or social media platform may be used to either promote the study or engage in patient participants providing feedback on treatment, success or, in some cases, failure. In some cases, completely open communication platforms (YouTube, twitter) may be integrated into the system.

The monitoring function used by the NFT blockchain can also be tied into the Internet ad network. See FNC 41. It is well known that the ad network provides funding for social media platforms as well as some websites by presenting ads to viewers of social media content CNT and web site content CNT.

FIG. 3A also shows that the blockchain function rewards a user when they complete the user personal profile UPP and the system assigns the unique name user U/N for the system. A password is assigned the UPP registered user. The passwords employed in the system can have hierarchical levels of access (that is different password access thresholds). One p/w permits a private or internal viewing of the blockchain network. A second password p/w to enables the user to post comments on the website supporting NFT blockchain. Participation points and clinical studies CLS 1, 2, 3, are documented by a blockchain link as function L-FNC 112 (FIG. 2A). CLS reports points are created by drafting the report, publishing the abstract of the report, publishing the entire report, and funding the NFT driven system.

The smart contract can give “participation points” to patients, which are akin to loyalty points. The loyalty points can be redeemed as free admission to educational seminars (these being utility tokens minted by the NFT blockchain, permitting admission to events, lectures, etc.); access to participate in the clinical study; and copies of the final report. The loyalty point program can be extended to any or all of the players and participants. The smart contract(s) can give % income shares (OIUs) to certain people (investors, clinicians who gather data; clinicians who draft the reports; clinicians who review/edit/approve the reports); and promoters (investors, clinicians who gather data; clinicians who draft the reports; persons who promote seminars during which the reports are discussed). There are many, many variations on the input and output of the smart contract. It is not feasible to outline all variations.

In FIG. 3A, points are captured in a point compiler database DB (the Complier 40) and the NFT has a threshold function converting points to OIUs. Compiler 40 is triggered by BLK-L11 in FIG. 2B. The points are locked into the NFT in a blockchain link or cell unit, BLK-L11.

FIG. 3B, 15 an example of point-to-ownership interest units (OIUs). At Level 1, column 1, an abstract interaction is shown. The NFT conversion function shows that 50 points are awarded for developing the abstract, and also shows the point to OIU conversion is 100 to 1. One hundred (100) points are needed for point conversion into one OIU. Educational participation units EPU, Level 2, in col. 2, are awarded to the registered or engaged participant for attendance at conferences, whether virtual or physical, and at seminars. Limited participation unit points PU-Pts (Level 3, col. 3) are awarded for interaction on committees. Participation PU points are also subject to the pre-programmed 100 pts-for-1 OIU in the NFT threshold.

Patient points PAT-pts (Level 4, col. 4) are awarded because patient data is needed to generate not only the first CLS report but also follow-on CLS reports. CL-pts points (Level 5, col. 5) are established both for patients who complete the entire CLS data input cycle and are involved in the clinical studies as well as clinician activity in the clinical studies. CLS points are awarded for drafting the clinic clinical studies (Levels 6 and 7, col. 6 and 7).

The smart contract for a given clinical collaboration may bestow rewards on patients that participate e.g., by providing feedback on their physical conditions and effect of the medical treatments administered on them in the trials.

As an incentive to participate in the CL study or promote the CLS abstract or CLS study even as a viewer, the point calculator in Level 8, (col. 8), shows an award of some points when the person or entity joins the CLS blockchain system. Once a user completes a an UPP in the system, see event 46 in FIG. 3A, the NFT CLS system can track such user and award points for viewing content CNT. Each new engaged or registered participant, per conversion function Level 8, is awarded 5 points. In Level 9 (col. 9), a CLS report funding or fiscal action is noted. Some persons may wish to fiscally invest in the NFT or the CLS report and this funding is rewarded by assignment of OIUs, via the point system by converting the amount of funds provided to the system and to a point-based system. The shown value in FIG. 3B notes that a fiscal contribution of $5,000 is equivalent to one OIU and this OIU is recorded in the NFT blockchain as an owner interest.

The system may tokenize points by minting a OIU NFT token or by giving a reward to participants (a utility token). These NFT awards could occur at intervals, upon specified events or milestones or time-frames, at the conclusion of the clinical trial, or after a final evaluation and allocation is made consistent with the smart contract rules. The smart contract establishes the rules for all participants based upon interactions of any nature: (i) issuance of OIU NFT tokens representing % income; (ii) issuance of loyalty or reward points; or (iii) giving a utility token (such as a “social club admission” or access to educational events) reward to qualifying participants. The term “NFT Participant” refers to all the players in the NFT clinical scheme. Any output from the NFT (that is, any reward/$/social club access), could occur at intervals, upon specified events locked into the NFT smart contract, or milestones monitored by the NFT smart contract, at the conclusion of the clinical trial, or after a final evaluation.

The following Abbreviations Table provides brief, but not complete, explanations of abbreviations used in the drawings.

Abbreviations Table

| | |
|---|---|
| Abstr . . . | Abstract of CLS Clinical Study Report |
| Abstr-Pts . . . | Points related to Abstract of CLS |
| BLK . . . | Blockchain |
| BLK-CLS . . . | BLK for Clinical Study Report (CLS Rpt) |
| BLK-CNT . . . | BLK with content (CNT), differentiate external/internal platform |
| BLK-L1, 2, 3 . . . | BLK Link1 or cell 1, Link2, Link3 |
| CL . . . | Clinic where studies are conducted, CL1, CL2, etc. |
| CL-BLK . . . | BLK Clinical Platform or System |
| CL-PAT-Pts . . . | CL Patient Points (participate in CL Study) (CL data) |
| CL-Pts . . . | Clinical Study HC Provider points |
| CLS 1, 2 . . . | Clinical Study Results 1, 2, sometimes CLS Rpt (published) |
| CNT . . . | content |
| CPI . . . | Cost per impression, normally 1,000 count units |
| DB . . . | Database, see master or Mstr DB |
| Dft . . . | Drafts as in draft CLS Report |
| DL .. | Distributed Ledger (DLT Distributed Ledger Technology) |
| EPU . . . | Education Units, e.g., seminars, PU committee/meeting action |
| EPU-Pts . . . | EPU points, different for attendees, participation, presenter |
| Ext . . . | External, as in available to the public |
| FNC . . . | Function, as in functional operations in blockchain, smart contract |
| HCP . . . | Healthcare (HC) Provider |
| Int . . . . | Internal or non-public |
| L-FNC . . . | Link or cell in Blockchain operating as a Function |
| MSTR DB . . . | Master Database |
| NFT . . . | non-fungible token, e.g., NFT blockchain or NFT owner token |
| Ntwk . . . | Network or platform |
| ORR . . . | Outside reviewer/critical commentor and ORR CNT |
| ORR-Pts . . . | Points for ORR and ORR CNT |
| ORR-CNT . . . | Content provided by ORR |
| P/W . . . | password |
| PAT . . . | Patient |
| PAT-Pts . . . | Patient points, e.g., CL-PAT points: participate in CL Study |
| PI . . . | Principal Investigator(s) for leading CL study |
| PI-Pts . . . | Principal Investigator(s) points |
| Pts-Suppl . . . | Points for Supplemental CNT or editing of CLS rpt |
| Pts . . . | Points, e.g., PU-Pts, EPU-Pts, Abstr-Pts, Access/View Pts for CLS |
| | CNT |
| PU . . . | Participant Units for interaction in CL, committees, etc. |
| PU-Pts . . . | PU Points: Rvw CLS, comment on CLS, etc. |
| Pub . . . | publish, as in publish the Abstract or the CLS Rpt |
| Req'd . . . | required, as in Participation required for investor-funder |
| Rpt . . . | Report |
| Rvw . . . | Review |
| SM . . . | Social Media |
| STAT . . . | Statistical threshold or Statistical analysis |
| Suppl-Pts . . . | Points for Supplemental CNT or editing of CLS rpt |
| t . . . | time, as in T1, 2, 3 is time period 1, 2, 3 |
| U/N . . . | User name unique to CL-BLK |
| UPP . . . | user personal profile data |
| Web . . . | Website 1, 2, 3, Web 3.0 refers to open-public NFT platform |

There are many ways of designing a point system for the clinical study. The following Chronologic Point Table expands upon the basic theories set forth above and sets forth additional participation points based upon the following Time Events: Gather Clinical Data at Clinic (CL); Draft CLS Rpt (CL Study (CLS) Rpt: internal action); Published Abstract (Abstr); Published CL Rpt on NFT external monitored platform; and CLS Rpt Published Internal Action on CLS NFT monitored platform. Hence, internal or on-platform interactions can occur at the same as external interactions.

Chronologic Point Table

| Time Event A: | Gather Clinical Data at Clinic (CL) |
|---|---|
| CL-PAT-Pts . . . | CL Patient Points (participant in CL Study) (CL data) |
| PAT-Pts . . . | Patient points, e.g., CL-PAT pts |
| PI-Pts . . . | Principal Investigator (PI) pts |
| HC-Pts . . . | Healthcare (HC) Provider—Clinical staff pts |
| Time Event B: | Draft CLS Rpt (CL Study (CLS) Rpt: internal action) |
| CL-Pts . . . | PI-Pts and HC-Pts for CLS Rpt 1st draft (dft 1), for dft 2, etc. |
| ORR-Pts . . . | Pts for internal review/revision (RR), Outside RR (ORR), and ORR |
| | CNT |
| Suppl-Pts . . . | Pts for Supplemental content (CNT) or editing CLS Rpt |
| PU-Pts . . . | Participation Unit (PU) Pts: RR, comment/email/blog/text/ |
| | broadcast, etc. |
| Time Event C: | Published Abstract (Abstr) |
| Abstr-Pts . . . | Pts for Abstr publication (to PI and HC Provider) |
| ORR-Pts . . . | Pts for external ORR and ORR CNT on Abstr |
| CL-Pts . . . | PI-Pts and HC-Pts for responding to Abstr inquiries |
| PU-Pts . . . | PU Pts: external broadcast Abstr, view/Rvw/comment/email/blog/ |
| | text, etc |
| Time Event D: | Published CL Rpt on NFT external monitored platform |
| PU-Pts . . . | PU Pts: external broadcast CLS Rpt, view/comment/email/blog/text, |
| | etc |
| CL-Pts . . . | PI-Pts and HC-Pts for responding to CLS Rpt inquiries |
| EPU-Pts . . . | Educational PU pts re CLS Rpt: differentiate participant/attendee/ |
| | presenter |
| PU-Pts . . . | PU Pts: broadcast Educ. Pgm .: Rvw/comment/email/blog/text/tweet |
| Time Event E: | CLS Rpt Published Internal Action on CLS NFT monitored platform |
| PU-Pts . . . | PU Pts: Internal re: CLS Rpt: view/Rvw/comment/email/blog/text/ |
| | tweet |
| EPU-Pts . . . | Internal Educ. Pgm. Pts., participant/attendee/presenter |
| PU-Pts . . . | PU Pts: Internal Educ. Pgm .: Rvw/comment/email/blog/text/tweet |
| CL-Pts . . . | PI-Pts and HC-Pts for responding to Educ. Pgm. inquiries |

The following Condensed Point and Abbreviations Table also has certain abbreviations used to differentiate points in the examples.

Condensed Point and Abbreviation Table
participation point (Pts)
abstract participation point (APP)
abstract interaction (IAI), may be
comment, edit, criticism, or distribution
clinical study report point (RPP)
clinical report interaction (IRI), may be
comment, edit, criticism, or distribution
external abstract participation point (xAPP)
external report interaction (ERI), may be
comment, edit, criticism, or distribution
general participation point (GPP)
general interaction (GI), may be comment,
edit, criticism, or distribution
patient participation point (PPP)
clinician participation point (CPP)
fiscal point (FP) to each fiscal participant
clinician Pts assigned to clinicians

FIGS. 4 and 5 diagrammatically illustrate high-level structures for blockchain NFT's for rewarding content creation, promotion and participation. In FIG. 4, sponsors 1, 2, 3 provide content (CNT) which is processed by the blockchain. The sponsor in event 50 provides content to the blockchain developer. In cell 51 of the blockchain, a function excepts the content and secures the content on the access control data structure. Additionally, the blockchain obtains user information from the sponsor and stores the same in the UPP. In cell 53, BLK-L201, the blockchain issues OIUs indicating ownership associated with the blockchain. Cell 55, BLK-L202, is a smart contract which monitors external promotion event 52 and network promotion of the entire blockchain as event 54. In other words, the promotion of the blockchain as a data structure itself, which limits access to content CNT, involves a different type of promotional event (event 54) in contrast to the external promotion of stored content CNT (event 52), which is more focused on promoting previously stored CNT via the storage process by the blockchain in cell 51 and event 50. In other words, the earlier stored content CNT has a scarcity characteristic because access to that earlier stored CNT is limited by the blockchain data structure. As described above, the blockchain data structure internally controls access to CNT.

Blockchain cell 55, BLK-L202, monitors external promotion event 52 in the network promotion event 54, engages in the conversion program discussed earlier, and issues OIUs based on the conversion of points. Points include internal or on-platform interactions, comments, reviews, internal distribution efforts, internal promotion of the CNT, etc. Points also include external or off-platform interactions, comments, reviews, external distribution efforts, external promotion of the CNT, etc. Cell 57, BLK-L203, and event 56 monitors internal interaction and assigns participation points to that internal interactional events by engaged or registered participants. Cell 59, BLK-L204, engages a full close out function or a partial close out function of the CNT. The CNT is subject to limited access by its functional relationship to the NFT and access controlled data structure. A full close out may be a complete sale of the CNT and release of all rights to that content. In this manner, as shown event the 58, the sale involves curated or popular content which has been vetted, reviewed, promoted and distributed potentially on a limited basis to NFT engaged participants. A partial close out would sell some of the rights the content CNT but retain some participation rights by existing NFT owners holding OIUs. As noted in FIG. 4, this may involve a fiscal contribution by the purchaser of those NFT platform rights.

FIG. 5 diagrammatically shows blockchain for curated content. Event 60 indicates that a sponsor has provided content CNT to the blockchain developer and, in blockchain cell 61, the content is subject to an imposed scarcity characteristic (that is, the CNT is isolated in the NFT data structure with little or no public access permitted). Additionally, NFT owner and participant data is stored in a UPP. Event 62 indicates that there is internal participation and curation of the content. This internal curation may include edits to the content, favorable or unfavorable reviews of the content, supplemental commentary or other refinements to the content. The NFT closed-access data structure stores the curated content. In cell 63, BLK-L301, initial NFT OIUs may be issued or minted. Event 64 recognizes external promotion of a preview of the curated content, which may include abstracts or the release of favorable commentary supporting a to-be-later-released version of the CNT. This external promotion activates the point compiler. Cell 65, BLK-L302, issues or mints OIUs based upon external participation points, interactions and events monitored by the blockchain smart contract or function. Stated otherwise, blockchain functions 63, 65 respectively monitor internal interactions and external interaction, collect and compile participation points, and, in some instances, issue NFT OIUs. Event 66 involves internal promotion of the curated CNT and the resulting internal interactions, and in cell 67, BLK-L303, issues internal participation points and, in some instances, issues or mints NFT OIUs.

Event 68 is the final curation of the CNT and cell 69, BLK-L304, monitors the final curation of the CNT. Event 70 and cell 71, BLK-L305, engages and executes a close out function. This close out function may involve an ad campaign based upon the internal data collection and the external data collection by the engaged or registered participants from events 62, 64, 66 and 68. The close out function in event 70 may have various characteristics as discussed above. Event 72 indicates that the content CNT and all rights to that CNT have been sold by the blockchain developer and the developer has received some fiscal renumeration. Cell 71, operating a smart contract, distributes that fiscal renumeration to NFT owners pro rata based upon their OIU ownership.

As used herein, the term “fiscal” refers to or relates to a financial aspect of the clinical study or the curated content CNT discussed in connection with FIGS. 4 and 5. Smart contracts are digital contracts stored on a blockchain that are automatically executed when predetermined terms and conditions are met. Some commentators use “smart contracts” to describe computer code that automatically executes all or parts of an agreement and is stored on a blockchain-based platform.

As for the Internet being a data structure, some commentators describe the Internet as a public data structure because data structures are the organizational tools data scientists use to update, manage and index internet services efficiently. Data structures are also algorithmic systems, due to the way the algorithmic systems filter and sort large quantities of data. There are three main parts to a data structure that make it work: (i) the Memory Address: The fixed raw data element of any desired feature or function; (ii) The Pointer: A reference tool that represents the location of a memory address; and (iii) The Procedure: A written code that manipulates or creates different functions inside the structure, either automatically or manually. The pointer, the memory address and the procedures of a data structure are important, as the use of them, or the way that they are represented in any given database or website, determines the different types of data structures a database can fall into.

As described above, the inventions relies upon and uses software or computer implemented embodiments. The terms “program,” “software application,” “APP” and the like as used herein, are defined as a sequence of instructions designed for execution on a computer system. A “program,” “computer program,” or “software application” may include a subroutine, a function, a procedure, an object method, an object implementation, an executable application, an applet, a servlet, a source code, an object code, a shared library/dynamic load library and/or other sequence of instructions designed for execution on a computer system. Those skilled in the art can understand the specific meanings of the above-mentioned terms in the embodiments of the present invention according to the specific circumstances. Further, although sequential steps are discussed in connection with the embodiments of the invention, the steps and processes may be re-ordered for efficiency or other reasons apparent to those skilled in the art. Additionally, although NFTs and blockchains are discussed in connection with embodiments of the present invention, the invention is not so limited to those types of data structures. Stated otherwise, a comprehensive computer program or groups of computer programs may be designed to fulfill the functions and processes described and claimed herein. In this sense, the invention is not limited to NFTs, blockchains, DLT systems or DAO systems.

The claims appended hereto are meant to cover modifications and changes within the scope of the present invention. What is claimed is:

1. A method of operating a data structure to support a clinical study with a plurality of patients, one or more healthcare clinicians, and one or more fiscal participants comprising:
each patient of the plurality of patients, each clinician of the one or more clinicians, and each fiscal participant of the one or more fiscal participants being an engaged participant;
providing a controlled data structure with access controls wherein access is provided for each engaged participant;
providing access to a public data structure;
assigning a participation point (Pts) based upon individual efforts of each engaged participant;
assigning clinician Pts to each clinician of the one or more clinicians for compiling clinical study data, and one or more of drafting an abstract, drafting a clinical study report, and editing;
assigning fiscal Pts to assigned each fiscal participant of the one or more fiscal participants based upon individual fiscal support of the clinical study;
after an internal distribution of the abstract on the controlled data structure, assigning an internal abstract participation point (APP) based upon individual efforts of engaged participants who have an abstract interaction (IAI) which is one or more of an IAI comment, an IAI edit, an IAI criticism, or an IAI distribution act;
after an internal distribution of the clinical study report on the controlled data structure, assigning an internal clinical study report point (RPP) based upon individual efforts of engaged participants who have a clinical report interaction (IRI) which is one or more of an IRI comment, an IRI edit, an IRI criticism, or an IRI distribution act;
releasing to the public data structure a public abstract subject to the internal distribution of the abstract;
assigning an external abstract participation point (xAPP) based upon individual efforts of the engaged participants who have an external abstract interaction (EAI) which is one or more of an EAI comment, an EAI edit, an EAI criticism, or an EAI distribution act;
releasing to the public data structure a public clinical study report subject to the internal distribution of the clinical study report;
assigning an external report participation point (xRPP) based upon individual efforts of the engaged participants who have an external report interaction (ERI) which is one or more of an ERI comment, an ERI edit, an ERI criticism, or an ERI distribution act;
assigning an ownership interest unit of a plurality of ownership interest units to the fiscal participant of one or more fiscal participants on a fiscal pro rata basis;
converting Pts, clinician Pts, fiscal Pts, APP, RPP, xAPP, and xRPP into one or more ownership units based upon a predetermined point-to-unit conversion;
providing a smart contract operating on the controlled data structure and the internet data structure;
determining success of the public clinical study report by the smart contract monitoring performance interest on the public data structure when performance interest exceeds a predetermined threshold; and
upon success, fiscally rewarding respective engaged participants based upon their ownership units.

2. The method of operating a data structure as claimed in claim 1 including assigning a patient participation point (PPP) to each patient of the plurality of patients for individual effort in the clinical study;
wherein clinician Pts are differentiated from Pts and from PPP assigned to each patient; and fiscal Pts are differentiated from both clinician Pts and PPP assigned to each patient.

3. The method of operating a data structure as claimed in claim 2 wherein the smart contract on the controlled data structure is communicatively coupled to the internal data structure and the public data structure.

4. The method of operating a data structure as claimed in claim 3 wherein the public data structure is an internet.

5. The method of operating a data structure as claimed in claim 4 wherein the controlled data structure includes one of a blockchain, a non-fungible token (NFT) blockchain, and a distributed ledger technologic (DLT) structure; and,
wherein prior to granting access to the controlled data structure, engaged participants provide personal identifying information (PII) to a user personal profile (UPP) data store in the controlled data structure and access to the controlled data structure is provided by a respective access pass assigned to each engaged participant.

6. The method of operating a data structure as claimed in claim 5 includes a plurality of non-participants who are interested in the clinical study as an incipient engaged participant; gathering PII for the UPP data store for each incipient engaged participant of the plurality of non-participants, and, once respective PII is uploaded to the UPP data store, each incipient engaged participant defined as one of the engaged participants;
assigning a general participation point (GPP) to each engaged participant (m) who interacts with the internally distributed abstract, (n) who interacts with the internally distributed clinical study report, (o) who interacts with the public abstract, and (p) who interacts with the public clinical study report, wherein a general interaction (GI) is one or more of a GI comment, a GI edit, a GI criticism, or a GI distribution act; and
differentiating GPP from clinician Pts, PPP, and fiscal Pts; and
wherein converting Pts into one or more ownership units includes converting GPP into one or more ownership units based upon the predetermined point-to-unit conversion.

7. A method of operating a data structure in support of a clinical study monitoring a plurality of patients engaged in the clinical study, and a creation, a review, an internal distribution, an external distribution, and a fiscal support of a clinical study report comprising:
providing a controlled data structure with access controls;
providing a public data structure for public release of a clinical study report based upon the clinical study;
granting access to the controlled data structure for a plurality of patients, and one or more healthcare clinicians, and one or more fiscal participants, collectively defined as registered users, having access to the controlled data structure via the access controls;
assigning a patient participation point (PPP) to each patient of the plurality of patients who participates in the clinical study based upon their individual patient participation level;
assigning a clinician participation point (CPP) to each clinician of the one or more clinicians who (i) compiles clinical study data from the plurality of patients, (ii) creates an abstract of the clinical study report, and (iii) creates the clinical study report, and further based upon a respective clinician participation level;
assigning a fiscal point (FP) to each fiscal participant of the one or more fiscal participants based upon individual fiscal support of the clinical study;
internally distributing the abstract on the controlled data structure;
assigning an internal abstract participation point (APP) (a) to each patient of the plurality of patients who interacts with the abstract and (b) to each fiscal participant of the one or more fiscal participants who interacts with the abstract, wherein an internal abstract interaction (IAI) is one or more of an IAI comment, an IAI edit, an IAI criticism, or an IAI distribution act;
internally distributing the clinical study report on the controlled data structure; assigning an internal report participation point (RPP) (a) to each patient of the plurality of patients who interacts with the clinical study report, and (b) to each fiscal participant of the one or more fiscal participants who interacts with the clinical study report, wherein an internal report interaction (IRI) is one or more of an IRI comment, an IRI edit, an IRI criticism, or an IRI distribution act;
after internally distributing the abstract, releasing to the public data structure a public abstract;
assigning an external abstract participation point (xAPP) (a) to each patient of the plurality of patients who interacts with the public abstract, and (b) to each fiscal participant of the one or more fiscal participants who interacts with the public abstract, wherein a public external abstract interaction (EAI) is one or more of an EAI comment, an EAI edit, an EAI criticism, or an EAI distribution act;
after internally distributing the clinical study report, releasing to the public data structure a public clinical study report;
assigning an external report participation point (xRPP) (a) to each patient of the plurality of patients who interacts with the public clinical study report, and (b) to each fiscal participant of the one or more fiscal participants who interacts with the public clinical study, wherein a public external report interaction (EM) is one or more of an ERI comment, an ERI edit, an ERI criticism, or an ERI distribution act;
assigning an ownership interest unit of a plurality of ownership interest units to the one or more fiscal participants on a fiscal pro rata basis;
converting PPP, CPP, FP, APP, RPP, xAPP, and xRPP into one or more ownership units based upon a predetermined point-to-unit conversion plan;
determining success of the public clinical study report by a smart contract on the controlled data structure which monitors performance on the public data structure and determines success based upon (I) acquired data representing public interaction with the public clinical study report and (II) one or more predetermined success thresholds; and
if success is determined, then fiscally rewarding respective registered users based upon their ownership units.

8. The method of operating a data structure in support of a clinical study as claimed in claim 7 wherein, after one or more of the internal distribution of the abstract, the internal distribution of the clinical study report, the release of the public abstract, and the release of the public clinical study report:
assigning an additive clinician participation point (xCPP) to each clinician of the one or more clinicians (w) who interacts with the internally distributed abstract, (x) who interacts with the internally distributed clinical study report, (y) who interacts with the public abstract, and (z) who interacts with the public clinical study report, wherein a clinician interaction (CI) is one or more of a CI comment, a CI edit, a CI criticism, or a CI distribution act.

9. The method of operating a data structure in support of a clinical study as claimed in claim 8 wherein the controlled data structure includes one of a blockchain, a non-fungible token (NFT) blockchain, and a distributed ledger technologic (DLT) structure, and wherein the public data structure is an internet, and,
wherein prior to granting access to the controlled data structure, the registered users provide personal identifying information (PII) to a user personal profile (UPP) data store in the controlled data structure.

10. The method of operating a data structure in support of a clinical study as claimed in claim 9 wherein assigning FP to each fiscal participant of the one or more fiscal participants precedes (i) assigning PPP to each patient of the plurality of patients and (ii) assigning CPP to each clinician of the one or more clinicians.

11. The method of operating a data structure in support of a clinical study as claimed in claim 10 includes engaging a plurality of incipient participants who are interested in the clinical study as registered users;
gathering PII for the UPP data store for each incipient participant of the plurality of incipient participants and further defining the incipient participant as a registered user;
after one or more of the internal distribution of the abstract, the internal distribution of the clinical study report, the release of the public abstract, and the release of the public clinical study report:
assigning a general participation point (GPP) to each registered user (m) who interacts with the internally distributed abstract, (n) who interacts with the internally distributed clinical study report, (o) who interacts with the public abstract, and (p) who interacts with the public clinical study report, wherein a general interaction (GI) is one or more of a GI comment, a GI edit, a GI criticism, or a GI distribution act.

12. The method of operating a data structure in support of a clinical study as claimed in claim 10 wherein only registered users have access to the controlled data structure.

13. A method of operating a data structure to support an operational clinical study with a plurality of patients, one or more healthcare clinicians, and one or more fiscal participants comprising:
each patient of the plurality of patients, each clinician of the one or more clinicians, and each fiscal participant of the one or more fiscal participants defined as an engaged participant;
masking personal identifying information (PII) of each patient of the plurality of patients from each patient's clinical study data obtained in the operational clinical study;
providing a controlled data structure which limits access to data stored in the data structure;
uploading each patient's clinical study data into the controlled data structure;
the controlled data structure provided with access to a public interne data structure (Internet) via a data communications module;
assigning a participation point (Pts), based upon individual efforts of each engaged participant;
for one or more of compiling clinical study data, drafting an abstract, drafting a clinical study report, and editing, assigning clinician Pts to each clinician of the one or more clinicians; assigning fiscal Pts to fiscal participants of the one or more fiscal participants for individual fiscal support of the operational clinical study;
after an internal distribution of the abstract on the controlled data structure to all engaged participants, assigning an internal abstract participation point (APP) based upon individual efforts of respective engaged participants who have an abstract interaction (IAI) which is one or more of an IAI comment, an IAI edit, an IAI criticism, or an IAI distribution act;
after an internal distribution of the clinical study report on the controlled data structure to all engaged participants, assigning an internal clinical study report point (RPP) based upon respective individual efforts of engaged participants who have a clinical report interaction (IRI) which is one or more of an IRI comment, an IRI edit, an IRI criticism, or an IRI distribution act;
releasing on the Internet a public version abstract;
assigning an external abstract participation point (xAPP) based upon individual efforts of the engaged participants who have an external abstract interaction (EAI) with the public version abstract which is one or more of an EAI comment, an EAI edit, an EAI criticism, or an EAI distribution act;
releasing on the Internet a public version clinical study report;
assigning an external report participation point (xRPP) based upon individual efforts of the engaged participants who have an external report interaction (ERI) with the public version clinical study report which is one or more of an ERI comment, an ERI edit, an ERI criticism, or an ERI distribution act;
assigning an ownership interest unit of a plurality of ownership interest units to the fiscal participant of one or more fiscal participants on a fiscal pro rata basis;
converting Pts, clinician Pts, fiscal Pts, APP, RPP, xAPP, and xRPP into one or more ownership units based upon a predetermined point-unit conversion;
providing a smart contract operating on both the controlled data structure and the Internet;
determining success of the public clinical study report by the smart contract monitoring performance interest on the Internet when performance interest exceeds a predetermined threshold; and
upon success, fiscally rewarding respective engaged participants based upon their ownership units.

14. The method of operating a data structure as claimed in claim 13 wherein clinician Pts are differentiated from Pts assigned to each patient of the plurality of patients who are engaged in the clinical study, and wherein fiscal Pts are further differentiated from both clinician Pts and Pts assigned to each patient. The method of operating a data structure as claimed in claim 14 includes a plurality of non-participants who are interested in becoming one of the engaged participants;
gathering respective PII for the UPP data store for each incipient engaged participant of the plurality of non-participants, and, once respective PII is uploaded to the UPP data store, each incipient engaged participant defined as an engaged participant;
assigning a general participation point (GPP) to each engaged participant (m) who interacts with the internally distributed abstract, (n) who interacts with the internally distributed clinical study report, (o) who interacts with the public abstract, and (p) who interacts with the public clinical study report, wherein a general interaction (GI) is one or more of a GI comment, a GI edit, a GI criticism, or a GI distribution act; and
differentiating GPP from clinician Pts, Pts assigned to each patient of the plurality of patients who is engaged in the clinical study, and fiscal Pts; and
converting Pts into one or more ownership units includes converting GPP into one or more ownership units based upon the predetermined point-unit conversion.
